(19) United States
(12) Patent Application Publication
(10) Pub. No.: US 2019/0175458 A1
(43) Pub. Date: Jun. 13, 2019

(54) PERCARBONATE IN A WATER-SOLUBLE FILM FOR BLEACHING HUMAN HAIR

(71) Applicant: Henkel AG & Co. KGaA, Duesseldorf (DE)

(72) Inventors: Udo Erkens, Willich (DE); Torsten Lechner, Langenfeld (DE)

(73) Assignee: Henkel AG & Co. KGaA, Duesseldorf (DE)

(21) Appl. No.: 16/212,265

(22) Filed: Dec. 6, 2018

(30) Foreign Application Priority Data
Dec. 7, 2017 (DE) 10 2017 222 124.0

(51) Int. Cl. A61K 8/11; A61Q 5/08; A61K 8/23; A61K 8/25; A61K 8/81; A61K 8/73; A61K 8/19

(52) U.S. Cl. CPC A61K 8/11; A61Q 5/08; A61K 8/23; A61K 8/25; A61K 8/8152; A61K 2800/5922; A61K 8/19; A61K 8/8129; A61K 8/732; A61K 2800/594; A61K 8/731

(57) ABSTRACT

The object of the present disclosure is a cosmetic agent for blonding human hair comprising
(i) a sachet that forms a closed chamber that comprises a water-soluble polymer, and
(ii) a bleaching composition, which is contained in closed chamber of the sachet, exemplified in that the bleaching composition contains
   at least one percarbonate and
   at least one persulfate.

### CROSS-REFERENCE TO RELATED APPLICATION

This application claims priority to German Patent Application No. 10 2017 222 124.0, filed Dec. 7, 2017, which is incorporated herein by reference in its entirety.

### TECHNICAL FIELD

The present disclosure relates to a cosmetic agent for dyeing human hair blond comprising (i) a sachet that forms a closed chamber and comprises a water-soluble polymer, and (ii) a bleaching composition which is contained in the closed chamber of the sachet. The present disclosure relates further to a method for dyeing human hair blond and the use of the cosmetic agent to dye human hair blond.

### BACKGROUND

To lighten or dye their hair blond or generally to make an oxidative colour change has been the desire of many consumers since time immemorial, because a blond hair colour is considered to be attractive and a worthwhile objective from a fashion point of view. For this purpose, various lightening agents with different lightening strengths are available on the market. The oxidising agents contains in these products are able to lighten the hair fibres by the oxidative destruction of the pigment melanin naturally present in the hair and/or of artificial dyes. For a moderate blonding effect, it is sufficient to use hydrogen peroxide alone as oxidising agent—possibly together with ammonia or other alkalising agents. To obtain a stronger blonding effect, usually a mixture of hydrogen—peroxide and peroxo salts, particularly persulfate salts is used.

These peroxo salts are typically used in the form of a powder, which is mixed with a hydrogen peroxide preparation shortly before it is applied. Use of the combination of hydrogen peroxide and persulfates is associated with various drawbacks. For example, application of hydrogen peroxide to the scalp can lead to irritation.

Furthermore, using hydrogen peroxide to lighten the hair entails a certain damage to the hair, since not only the dyes but also the other structural components of hair are damaged by oxidation. Depending on the nature of this degree of damage, it may take any form from coarse, brittle hair which is difficult to comb, to reduced resilience or tensile strength and split ends, and ultimately even hair breakage. The larger the quantity of hydrogen peroxide and peroxodisulfates used, as a rule the greater the damage the keratin fibre suffers. Thus it is also still a permanent challenge to find new lightening agents that are less damaging to hair. For the purposes of the present disclosure, the term lightening agent is understood to be synonymous with bleaching agent.

In order to produce the application-ready lightening agent, at least two separately packaged components, the persulfate powder and the hydrogen peroxide solution, must be mixed together. The sustainability-minded user is also paying closer attention to the ecological aspects of a given product. One objective in this context is also to reduce the amount of packaging material used. Products which are used in the most concentrated form possible, which include only one component, and which only need to be mixed with water to produce the application mixture optimally offer a decisive advantage with regard to saving packaging material.

Pre-dosed, packaged application mixtures are known in the related art. Water-soluble polymer films are used for example as packaging material for pre-measured liquid detergents or liquid dishwashing agents. They are packaged in films and available to the user as a one-time application in this form. The user can then place these sachets, pouches or pads directly in the washing machine or dishwasher. During use, the film dissolves, releasing its contents. For the user, this form of measured delivery offers several different advantages. The risk of excess dosing is avoided, and when the film dissolves completely the user does not have to dispose of it separately. Consequently, this form of measured delivery and application is particularly convenient for the user.

In the field of cosmetics, pre-dosed packaging in the form of a sachet poses very particular requirements for the formulation in the sachet. The aim is to provide only one sachet with one chamber, in other words not to package dyeing agents or precursors thereof in a first sachet and hydrogen peroxide in a second sachet. The use of such kits including of two different sachets is known and has drawbacks. During application in the form of a sachet, it must be ensured that the lightening effect is sufficiently powerful even without separate hydrogen peroxide. It must also be ensured that the contents do not enter into any cross-reactions with film that might impair the properties of the film. This represents a particular challenge the given context, because usual lightening agents contain alkalising agents as well as a hydrogen peroxide precursor, wherein this precursor must not be converted into hydrogen peroxide by moisture getting into the sachet while it is being stored, and this limits the shelf life of the cosmetic product.

The problem underlying the present disclosure is in providing a cosmetic blonding agent which guarantees a safe dosage, provides sufficient lightening effect without damaging the hair excessively, and is stable during storage.

### BRIEF SUMMARY

Cosmetic agents and methods for blonding human hair are provided herein. In an embodiment, a cosmetic agent for blonding human hair includes a sachet forming a closed chamber and a bleaching composition which is included inside the closed chamber of the sachet. The sachet includes a water-soluble polymer. The bleaching composition includes at least one percarbonate and at least on persulfate.

In another embodiment, a cosmetic agent for blonding human hair includes a sachet forming a closed chamber and a bleaching composition which is included inside the closed chamber of the sachet. The sachet includes a water-soluble polymer. The water-soluble polymer includes one or more polyvinyl alcohol polymers having an average molecular weight M_{w} from about 30,000 to about 170,000 g/mol. The bleaching composition includes sodium percarbonate or potassium percarbonate in a quantity from about 6 to 10% w/w relative to the total weight of the bleaching composition. The bleaching composition further includes sodium persulfate in a quantity from about 30 to about 45% w/w relative to the total weight of the bleaching composition. The bleaching composition further includes an alkalising agent chosen from sodium silicate, potassium silicate or a mixture of sodium silicate and potassium silicate. The alkalising agent is present in the bleaching composition in a quantity from about 35 to about 45% w/w relative to the total weight of the bleaching composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The problem underlying the present disclosure is solved with the object of Claim 1. A first object of the present disclosure is therefore a cosmetic agent for blonding human hair comprising (i) a sachet forming a single closed chamber which comprises a water-soluble polymer, and (ii) a bleaching composition which is contained in the closed chamber of the sachet, wherein the bleaching composition contains at least one percarbonate and at least one persulfate.

As contemplated herein, the cosmetic agent comprises a sachet and a bleaching composition, wherein the sachet contains the bleaching composition. The material from which the sachet is made comprises a water-soluble polymer. This facilitates convenient handling of the cosmetic agent. All that needs to be done is to mix the sachet containing the bleaching composition with water. The homogeneous mixture created thereby can then be applied to human hair.

Percarbonates and persulfates that are generally suitable for use as lightening agents are typically solid substances. The percarbonate and that persulfate which are used in the cosmetic agent as contemplated herein are preferably solids, particularly solids in powder form. The fact that the bleaching composition is contained in the sachet ensures that the bleaching composition is easily manageable and can be delivered reliably in measured quantities. The percarbonate used and the persulfate used do not generate dust, because they are contained inside the closed chamber in the sachet. Thus, the sachet of the cosmetic agent as contemplated herein thus forms a closed chamber. This may be guaranteed by sealing the chamber or closing it with adhesive after the filling the sachet with the bleaching composition.

The cosmetic agent as contemplated herein is used to change the colour of human hair oxidatively. The term “oxidative colour change” is understood to refer to blonding agents and also agents for lightening the colour of keratin fibres which contain the oxidising agents percarbonate and persulfate. If the intended result is simple blonding or lightening, the cosmetic agents contain no other dyes. However, the intention may be to impart tinting to the keratin fibres in addition to the blonding/lightening effect. For the purpose of tinting, the cosmetic agents as contemplated herein may also contain additional colouring components such as direct dyes and/or precursor products of oxidising dyes. However, the preferred purpose of the cosmetic agents is blonding or lightening, which is why the cosmetic agents preferably contain either no dyes or only very small quantities thereof, suitable only for slight tinting.

The product as contemplated herein includes a sachet which forms a closed chamber and contains a water-soluble polymer. In other words, the product as contemplated herein comprises a sachet (pouch) which forms at least one sealed chamber. The wall of the pouch (and thus of the chamber too) is produced from a water-soluble film. Thus, the closed chamber of the sachet comprises at least one water-soluble polymer.

The essential feature in this arrangement is that the film (or the water-soluble polymer from which the film is produced) is soluble in water, so the pouch dissolves during preparation of the application mixture.

In this context, water-solubility is determined according to the following method:

### Equipment

### Beaker (600 ml)

Magnetic stirrer (e.g., Labline model No. 1250 or equiv.) and agitator, 5 cm

### Thermometer (0-100° C.)

### Stopwatch (0-300 s)

Polaroid 35 mm slide holder
Clamps and stand
Distilled water (about 20° C.)

### Method

Pieces with dimensions of 3.8×3.2 cm are cut out of the film whose solubility in water is to be determined. These pieces are placed in the slide holder. The beaker is filled with 500 ml distilled water. The fill level in the glass is marked on the beaker. Then, the beaker is secured on the magnetic stirrer, the agitator is added and the magnetic stirrer is set to level at which a vortex is created in the beaker, so that the vortex reaches a fifth of the original fill level. The slide holder with the film attached is inserted in the beaker with the clamps in such manner that the long end of the slide holder is aligned parallel to the water surface. At the same time, the slide holder should be dipped into the water to a depth at which the top edge of the slide holder is 0.6 cm below the surface of the moving water. The short side of the slide holder should be positioned next to the wall of the beaker, and the other side should be aligned directly above the agitator.

The stopwatch is stared as soon as the slide holder is immersed in the water. The film disintegrates when the film breaks. As soon as all visible parts have become detached from the slide holder, the slide holder is removed from the beaker. Dissolution is complete as soon as no more film fragments are visible and as soon as the solution is clear again.

A film is soluble in water for the purposes of the present disclosure if it has dissolved according to the method described above within about 300 seconds (measurement conducted at 20° C.).

A polymer is soluble in water for the purposes of the present disclosure if it has dissolved according to the method described above within about 300 seconds (measurement conducted at 20° C.).

The film is preferably dissolved during the measurement according to the method described above within about 250 seconds, more preferably within about 200 seconds and particularly preferably within about 150 seconds.

According to a preferred embodiment of the present disclosure, the cosmetic agent comprises one or more polyvinyl alcohol polymers (PVOH polymers) as the water-soluble polymer.

As part of a further embodiment, the water-soluble polymer may also comprise a first PVOH-polymer and a second PVOH polymer, wherein the first PVOH polymer has a lower average molecular weight M_{w} than the second PVOH polymer and/or wherein the first PVOH polymer has a lower degree of hydrolysis than the second PVOH polymer.

Polyvinyl alcohol is a thermoplastic plastic which is usually manufactured by saponification (hydrolysis) of polyvinyl acetate (PVAC). The direct synthesis method, i.e. by polymerisation of vinyl alcohol, is not possible. When polyvinyl acetate is hydrolysed, it is unlikely that all acetate groups will be split even under powerful reaction conditions, so strictly speaking polyvinyl alcohols are copolymers. In the context of the present disclosure, a homopolymer a polyvinyl acetate that undergoes hydrolysis will be referred to as a PVOH polymer. A copolymer that is produced by polymerisation of vinyl acetate with a second monomer which is not vinyl acetate followed by hydrolysis will be referred to in the context of the present disclosure as a PVOH copolymer.

The properties of PVOH polymers are determined substantially by their molecular weight and degree of hydrolysis. The molecular weight of polymers affects cohesion in the solid polymer. The greater the molecular weight is, the greater the cohesion is as well. With greater cohesion, the adhesive strength when the sachet is closed and the stability of the sachet also increase. The degree of hydrolysis represents the amount of hydrolysed monomer units in the polymer as a fraction of all monomer units. If the degree of hydrolysis is equal to about 90%, 9 out of 10 monomer units in the PVOH polymer are saponified and one monomer unit represents a vinyl acetate unit. The degree of hydrolysis strongly influences the solubility of the polymers in water. The use of a PVOH polymer which is optimised in terms of molecular weight and degree of hydrolysis makes it possible to adjust both the stability of the sachet and the solubility properties of the sachets to a balanced degree at the same time.

When bleaching agents are packaged in pouches or sachets, the materials of the sachets (pouches) are subject to very particular requirements. The oxidising agents used to achieve the bleaching or blonding effects, such as persulfates for example, are aggressive reagents which not only turn hair or keratin fibres blond according to their purpose, but can also damage or break down the packaging materials by oxidation. Pouches which contain oxidising agents such as persulfates and percarbonates must therefore be very particularly resistant to chemicals, particularly oxidative influences.

In the course of the work which led to this present disclosure, it was found that the average molecular weight of the water-soluble polymer also seemed to have an influence on its chemical resistance.

According to a more preferred embodiment of the present disclosure, the at least one PVOH polymer has an average molecular weight M_{w} from about 30,000 to about 170,000 g/mol, preferably from about 40,000 to about 120,000 g/mol, more preferably from about 50,000 to about 120,000 g/mol and most particularly preferably from about 60,000 to about 110,000 g/mol (molar mass averages are given).

The best resistance to oxidative influences was observed when the water-soluble polymer used to produce the pouch had an average molecular weight M_{w} from about 30,000 to about 170,000 g/mol, preferably from about 40,000 to about 120,000 g/mol, more preferably from about 50,000 to about 120,000 g/mol and most particularly preferably from about 60,000 to about 110,000 g/mol.

In the context of the present disclosure, the average molecular weight M_{w} is always taken to be the middle weight average of the molecular weight. This is determined by gel permeation chromatography (GPC). An aqueous solution of 0.2 M NaNO₃ and 0.01 M NaH₂PO₄ at pH 7 is used as the eluent. The PL aquagel-OH column manufactured by Agilent Technologie is used as the chromatography column, and determination is carried out in accordance with brochure 5991 5780 EN published by Agilent Technologies in 2017. Any deviations in the determination of the molecular weights which are attributable to the determination method only affect the accuracy with which the average molecular weight can be determined. As a rule, the average molecular weight can be determined with sufficient accuracy using the GPC method if the elution agent is specified.

The degree of hydrolysis (or also degree of deacetylation) may be determined for example by measuring the polymer with quantitative 1H-NMR and/or 13C-NMR spectroscopy and comparing it with a fully acetylated or deacetylated reference polymer or another suitable standard.

The degree of hydrolysis of the polyvinyl alcohols have values from about 75% to about 99% for example. In this context, the percentages mean that about 75% of the vinylacetate units have been hydrolysed and transformed into the corresponding hydroxy groups. The degree of hydrolysis is preferably in the range from about 79 to about 92%, the degree of hydrolysis is most particularly in the range from about 82 to about 99%.

According to a preferred embodiment of the present disclosure, the water-soluble polymer includes one or more PVOH polymers in the amount of about 70% w/w, preferably at least about 80% w/w and more preferably at least about 85% w/w relative to the total weight of the sachet. In the context of the present disclosure, the term sachet always refers to the empty sachet. The material of which the sachet is made of the one or more PVOH polymers in the proportions described above. Plasticisers, auxiliary softeners or other polymer components are added as additional constituents.

According to another preferred embodiment, the water-soluble polymer further comprises a polyvinyl alcohol copolymer (PVOH copolymer) and/or a polysaccharide selected from the group of methyl cellulose, carboxymethyl cellulose, ethyl cellulose, hydroxyethyl cellulose, hydroxypropyl cellulose, hydroxypropyl methyl cellulose, dextrin and hydroxypropyl starch, wherein a water-soluble polysaccharide comprising a hydroxypropyl starch is particularly preferred. These polymers are the additional polymer constituents described above which may be used for preference as material for the sachet besides the one or more PVOH polymer(s).

In order to produce the at least one sealed chamber of the sachet (pouch), the water-soluble film described previously is used, wherein the film comprises the polymers described previously.

In this context, for example first the polymers are blended with each other—optionally with the application of heat—to produce the polymer blend. Then, the film can be formed from the polymers or polymer blend, wherein this forming may be carried out by casting, extrusion, rolling, for example, or similar process.

Then, a pouch is formed from this film by creating at least one sealed chamber in the film. The chamber may be created according to the methods known to the person skilled in the art. For example, preforms may first be created from the water-soluble film. These are then exposed to blowing pressure, wherein the preform is forwarded to various processing stations inside a blow moulding machine. Typically, such a blow moulding machine is equipped with a heating device and a blowing device, in the vicinity of which the previously temperature-controlled preform is expanded to form a container by biaxial stretching. The expansion process is assisted with compressed air, which is introduced into the preform that is to be expanded. In one embodiment, the chamber is filled with the shampoo and sealed as it is being formed.

Sealing may be carried out for example by fusing and pressing on the seams of the pouch.

These polymers may have a beneficial effect in the cosmetic agent. Particularly the hydroxypropyl starch functions as a thickener in the cosmetic agent. Given the consistency of the hydroxypropyl starch, it is disadvantageous if it is provided in the sachet as a part of the bleaching composition. As a part of the material which forms the sachet, the polymer constituents may be made available to the cosmetic agent advantageously by mixing it with water so that the sachet dissolves and releases the further polymer constituent. In particular, it is advantageous to use polymer constituents in the film which react when added to the alkalising agent separately from the bleaching composition.

According to a preferred embodiment of the present disclosure, the bleaching composition contains sodium percarbonate or potassium percarbonate in a quantity from about 0.5 to about 14% w/w, preferably from about 1.5 to about 13% w/w, more preferably from about 3 to about 12% w/w, still more preferably from about 4.5 to about 11% w/w and most particularly preferably from about 6 to about 10% w/w relative to the total weight of the bleaching composition as the percarbonate, and one or more of the group of ammonium persulfate, potassium persulfate and sodium persulfate in a quantity from about 5 to about 60% w/w, preferably from about 10 to about 55% w/w, more preferably from about 15 to about 50% w/w, still more preferably from about 20 to about 48% w/w and most particularly preferably from about 30 to about 45% w/w relative to the total weight of the bleaching composition as the persulfate.

For the purpose of the present disclosure sodium percarbonate is understood to refer to the adduct (or complex) of sodium carbonate and hydrogen peroxide with composition 2 Na₂CO₃×3 H₂O₂. Sodium percarbonate forms a white, water-soluble powder which disintegrates completely into in sodium carbonate and hydrogen peroxide on contact with water. The sodium percarbonate (2 Na₂CO₃×3 H₂O₂) as contemplated herein has a molar mass of 314.02 g/mol and has is given CAS number 15630-89-4.

Sodium percarbonate is available commercially in various degrees of purity from a number of suppliers. For example, Evonik Degussa offers a sodium percarbonate with a purity of about 98.8% w/w. All the aforementioned quantity specifications are indicated relative to 100% sodium percarbonate. If sodium percarbonate with lower degrees of purity is used, the quantities for use must be recalculated correspondingly.

Similarly, for the purpose of the present disclosure potassium percarbonate is understood to refer to the adduct (or complex) of potassium carbonate with hydrogen peroxide having the composition 2 K₂CO₃×3 H₂O₂.

The use of sodium percarbonate has been found to be particularly well suited for solving the problem as stated as contemplated herein.

It was found that hair damage could be reduced when smaller quantities of percarbonates than is conventionally the case were used in the cosmetic agent. In this context, it was surprising that—if the optimal content of percarbonates was selected and the percarbonates were introduced in certain quantitative proportions to the persulfates—this reduction in hair damage could be achieved without any loss of the blonding effect.

The research that resulted in this present disclosure showed that a further increase in the quantity of percarbonate above about 14% w/w is more damaging to the hair but does not further enhance the lightening effect. In this context, it was found to be preferable to use one or more percarbonates in a total quantity from about 1.5 to about 13% w/w, more preferably from about 3 to about 12% w/w, still more preferably from about 4.5 to about 11% w/w and most particularly preferably from about 6 to about 10% w/w in the cosmetic agents as contemplated herein. The best lightening effect with the comparatively least hair damage was obtained when the cosmetic agents contained the percarbonates (particularly the sodium percarbonate) in a total quantity from about 6 to about 10% w/w.

All the aforementioned specifications in % w/w in the context of the present disclosure are indicated relative to the total weight of the bleaching composition or the total weight of the cosmetic agent, depending on the indication. If a mixture of sodium percarbonate and potassium percarbonate is used, of course the specifications in % w/w relate to the sum of the weight percentages. Of course, the same applies for the persulfates.

As the second constituent of the bleaching composition which is essential to the present disclosure, the cosmetic agent contains one or more persulfates from the group of ammonium persulfate, potassium persulfate and sodium persulfate in a total quantity from about 5 to about 60% w/w, preferably from about 10 to about 55% w/w, more preferably from about 15 to about 50% w/w, still more preferably from about 20 to about 48% w/w and most particularly preferably from about 30 to about 45% w/w relative to the total quantity of the bleaching composition.

It has further proven to be advantageous if the cosmetic agent contains at least two persulfates from the group of ammonium persulfate, sodium persulfate and potassium persulfate. It is most particularly preferable if the cosmetic agent as contemplated herein contains all three persulfates—ammonium persulfate, sodium persulfate and potassium persulfate. It is particularly preferable if the cosmetic agent as contemplated herein contains from about 3 to about 20% w/w, preferably from about 5 to about 18% w/w, more preferably from about 7 to about 16% w/w and most particularly preferably from about 9 to about 14% w/w ammonium persulfate.

Ammonium persulfate is also referred to as ammonium peroxodisulfate, and has the molecular formula (NH₄)₂S₂O₈. Ammonium persulfate has CAS number 7727-54-0. Potassium persulfate is also called potassium peroxodisulfate and has the molecular formula K₂S₂O₈. Potassium persulfate has CAS number 7727-21-1. Sodium persulfate is also called sodium peroxodisulfate and has molecular formula Na₂S₂O₈. Sodium persulfate has CAS number 7775-27-1.

The persulfates are preferably also used in certain total quantities in the cosmetic agent as contemplated herein, to both optimise the lightening effect and minimise hair damage.

According to a further preferred embodiment of the present disclosure, the bleaching composition also contains an alkalising agent, which is preferably an alkaline or alkaline earth silicate, which is more preferably sodium silicate, potassium silicate or a mixture of sodium silicate and potassium silicate.

Das cosmetic agent is preferably constituted in such manner that the application-ready cosmetic agent obtained by mixing with water has an alkaline pH value. The application-ready cosmetic agent preferably has a pH value from about 8 to about 12, particularly preferably a pH value from about 8.5 to about 11, most especially preferably a pH value from about 9.5 to about 10.5, measured in each case at 20° C. For this measurement, the cosmetic agent is resuspended in a double quantity of water and the pH value is measured with a standard commercial pH-meter at the indicated temperature.

Percarbonates, particularly sodium percarbonate, dissolve in water, yielding a basic pH value. Depending on the total quantity of the percarbonates used in the cosmetic agent, the alkaline pH value in the application-ready cosmetic agent may therefore be created just with the percarbonates alone.

Particularly if the percarbonates are used in smaller quantities, or if the application-ready cosmetic agent is to have a particularly alkaline pH value, it may be advantageous to work an alkalising agent into the cosmetic agent as well. Since the bleaching composition is preferably prepared in the form of a powder, particularly suitable alkalising agents are solid at room temperature.

The alkalising agents that are usable as contemplated herein are more preferably selected from the group of alkaline (earth) metal metasilicates, alkaline (earth) metal hydroxides, alkaline (earth) metal phosphates, alkaline (earth) metal hydrogen phosphates and basic amino acids. Preferred alkali metal ions are lithium, sodium and/or potassium. Preferred alkaline earth metal ions are magnesium and/or calcium.

Further suitable alkalising agents are the basic amino acids such as arginine, histidine and lysine and/or salts thereof. Of the salts of arginine, histidine and lysine, those that are preferred as contemplated herein are the ammonium salts, alkali metal salts and alkaline earth metal salts, particularly the lithium, sodium, potassium, magnesium and calcium salts. The amino acids that are suitable as contemplated herein, selected from arginine, histidine and lysine, may also contain water of crystallisation.

The quantities of the one or more alkalising agent(s) used are chosen by the person skilled in the art depending on the pH value that is to be set in the application-ready cosmetic agent. However, it is preferable if the alkalising agent is contained in the bleaching composition in a quantity from about 20 to about 50% w/w, most preferably in a quantity from about 35 to about 45% w/w, relative in each case to the total weight of the bleaching composition.

According to a particularly preferred embodiment of the present disclosure, the bleaching composition contains a mixture of ammonium persulfate, potassium persulfate and sodium persulfate as the persulfate, in which mixture ammonium persulfate is present in a quantity from about 1 to about 15% w/w, potassium persulfate in a quantity from about 0 to about 45% w/w and sodium persulfate in a quantity from about 0 to about 5% w/w, with the general condition that the total of the quantities of ammonium persulfate, potassium persulfate and sodium persulfate is between about 15 to about 65% w/w, wherein the bleaching composition contains sodium percarbonate in a quantity from about 1 to about 10% w/w as the percarbonate, and wherein the bleaching composition contains sodium silicate in a quantity from about 35 to about 45% w/w as the alkalising agent, the quantities being relative to the total weight of the bleaching composition in each case. In this particularly preferred embodiment of the present disclosure, the best compromise between solubility of the constituents of the bleaching composition and blonding effect of the bleaching composition is attained with an advantageous pH value.

The bleaching composition in the cosmetic agent is particularly preferably constituted in such manner that when the bleaching composition is dissolved or suspended in a double quantity of water—relative to the weight of the bleaching composition—it has a pH value from about 8 to about 12, preferably from about 9.5 to about 10.5.

The bleaching composition exists in the form of a single component which simply has to be mixed with water in order to prepare the application-ready cosmetic agent. With this method, there is no need to mix it with a second, separately packaged preparation, and packaging material and the costs associated therewith may be saved. When mixed with water, hydrogen peroxide (or “active oxygen”) is released from the percarbonates in situ. Since the contact with water transforms the cosmetic agent into its application-ready form, the cosmetic agent itself is substantially anhydrous, thus containing less than about 10.0% w/w water. For example, 100 g of an agent as contemplated herein contain note more than about 9.9% w/w (=9.9 g) water.

However, various raw materials may contain small quantities of water, for example if they are utilised in the form of emulsions, contain water of crystallisation or if water is present as a minor constituent. If these raw materials are used, it is therefore possible for smaller quantities of water to be entrained into the bleaching composition. However, it is particularly advantageous to keep the water content as low as possible.

According to a preferred embodiment of the present disclosure, the bleaching composition further contains a dispersing copolymer based on acrylates, wherein the dispersing copolymer preferably comprises a copolymer containing one or more acrylic acid or methacrylic acid units, wherein the dispersing copolymer is more preferably a methacrylic acid/methyl(meth)acrylate copolymer or an acrylic acid/methyl(meth)acrylate copolymer.

As contemplated herein, the cosmetic agent is to be transformed into an application-ready agent by mixing it with water. When powders are mixed with water, it can happen that lumps or clumps are formed, while other parts of the powder are still not wetted by the water. Wetting with water can be difficult generally. A disperser is added to improve wetting. The copolymers listed previously have proven to be particularly advantageous.

According to a preferred embodiment of the present disclosure, the bleaching composition further contains a thickener which is selected from the group of a carboxymethyl cellulose, the Na salt thereof and hydroxymethyl cellulose. On the one hand, thickeners should be present in the bleaching composition largely as a pourable powder, and on the other in the application-ready cosmetic agent they should also raise the viscosity of the application-ready cosmetic agent to a user-friendly range as soon as possible after mixing with water. The thickeners specified above have proven to be the most advantageous in this regard.

A second object of the invention present disclosure is therefore a method for dyeing human hair blond, in which (a) the cosmetic agent as contemplated herein is introduced into a quantity of water, (b) the mixture thus obtained from (a) is homogenised, and (c) the homogenised mixture is applied to the human hair.

As was explained in detail earlier, the advantage of the present disclosure is that a single packaged object is provided, to which water is added so that the sachet dissolves and the bleaching composition is suspended, and the homogenised mixture is then present as an application-ready blonding composition. After it has been applied, the homogenised mixture is allowed to take effect, and finally rinsed out of the hair with water. In this way, the hair colour is made lighter.

According to a preferred embodiment of the present disclosure, the cosmetic agent according to the first object of the present disclosure may be supplied in a container, a bottle or a can. The dimensions of the container may be selected such that the container is only partly filled, so that the remaining capacity can be filled with water. The quantity of water which must then be added in order to prepare the application-ready cosmetic agent may be indicated for example by a marking on the wall of the container.

According to a preferred embodiment of the present disclosure, the mixing ratio of cosmetic agent as contemplated herein and water may be in the range from about 1:5 (1 part by weight of agent as contemplated herein for 3 parts by weight of water) to about 3:1, preferably from about 1:2 to about 1:1.

The application-ready cosmetic agents prepared from the cosmetic agent and water preferably have a viscosity in the range from about 3000 to about 40000 mPas, preferably about 4000 to about 30000 mPas, particularly preferably from about 6000 to about 15000 mPas, measured in each case at 20° C. with a Haake-cylinder/cylinder rheometer, SV I rotary/measurement system with a cooling time of about 5 minutes. With this measurement method, the viscosity value is determined for a shear rate of 1/7.2 s. The measurement programme works with a ramp of 0- 1/60 s. A viscosity in this range enables the application-ready cosmetic agent to be applied well, and also lends it a flow behaviour such that it the application site is exposed to the agent for long enough to guarantee that it will have the intended effect on the keratinous fibres.

The exposure time after step c) described above is preferably from about 5 to about 60 min, particularly from about 5 to about 50 min, particularly preferably from about 10 to about 45 min. During the exposure time for the homogenised mixture on the hair, it may be advantageous to support the dyeing process by adding heat. The exposure phase at room temperature also falls within the scope of the present disclosure. In particular, the temperature during the exposure time is between about 20° C. and about 40° C., particularly between about 25° C. and about 38° C. The agents already deliver good treatment results at physiologically tolerable temperatures of below about 45° C.

After the end of the exposure phase, all components remaining on the keratin fibres are rinsed out of the hair with water or a tenside-containing cleaning agent. In this context, cleaning agents may particularly be standard commercial shampoo, wherein the cleaning agent may particularly be omitted and the rinsing process may be carried out with tap water if the dyeing agent has a higher tenside content.

A third object of the present disclosure in therefore a use of a cosmetic agent as contemplated herein for for dyeing human hair blond.

As contemplated herein, most particularly preferred cosmetic agents comprise at least one of the following embodiments A) to G):

### A)

Cosmetic agent for blonding human hair comprising (i) a sachet which forms a closed chamber which contains a water-soluble polymer comprising one or more polyvinyl alcohol polymers (PVOH polymers), and (ii) a bleaching composition which is contained inside the closed chamber of the sachet, wherein the bleaching composition contains at least one percarbonate and at least one persulfate.

### B)

Cosmetic agent for blonding human hair comprising (i) a sachet which forms a closed chamber which contains a water-soluble polymer comprising one or more PVOH polymers and (ii) a bleaching composition which is contained inside the closed chamber of the sachet, wherein the bleaching composition contains at least one percarbonate and at least one persulfate.

### C)

Cosmetic agent for blonding human hair comprising (i) a sachet which forms a closed chamber which contains a water-soluble polymer comprising one or more PVOH polymers, wherein the first PVOH polymer has an average molecular weight M_{w} from about 30,000 to about 170,000 g/mol, preferably from about 40,000 to about 120,000 g/mol, more preferably from about 50,000 to about 120,000 g/mol and most particularly preferably from about 60,000 to about 110,000 g/mol and (ii) a bleaching composition which is contained inside the closed chamber of the sachet, wherein the bleaching composition contains at least one percarbonate and at least one persulfate.

### D)

Cosmetic agent for blonding human hair comprising (i) a sachet which forms a closed chamber which contains a water-soluble polymer comprising one or more PVOH polymers, and (ii) a bleaching composition which is contained inside the closed chamber of the sachet, wherein the bleaching composition contains sodium percarbonate or potassium percarbonate in a quantity from about 0.5 to about 14% w/w, preferably from about 1.5 to about 13% w/w, still more preferably from about 3 to about 12% w/w, particularly preferably from about 4.5 to about 11% w/w and most particularly preferably from about 6 to about 10% w/w relative to the total weight of the bleaching composition as the percarbonate, and one or more of the group of ammonium persulfate, potassium persulfate and sodium persulfate in a quantity from about 5 to about 60% w/w, preferably from about 10 to about 55% w/w, more preferably from about 15 to about 50% w/w, still more preferably from about 20 to about 48% w/w and most particularly preferably from about 30 to about 45% w/w, relative to the total weight of the bleaching composition, as the persulfate.

### E)

Cosmetic agent for blonding human hair comprising (i) a sachet which forms a closed chamber containing a water-soluble polymer comprising one or more PVOH polymers, wherein the first PVOH has a lower average molecular weight M_{w} than the second PVOH polymer and/or wherein the first PVOH polymer has a lower degree of hydrolysis than the second PVOH polymer, and (ii) a bleaching composition which is contained inside the closed chamber of the sachet, wherein the bleaching composition contains sodium percarbonate or potassium percarbonate in a quantity from about 0.5 to about 14% w/w, preferably from about 1.5 to about 13% w/w, still more preferably from about 3 to about 12% w/w, particularly preferably from about 4.5 to about 11% w/w and most particularly preferably from about 6 to about 10% w/w relative to the total weight of the bleaching composition as the percarbonate, and one or more of the group of ammonium persulfate, potassium persulfate and sodium persulfate in a quantity from about 5 to about 60% w/w, preferably from about 10 to about 55% w/w, more preferably from about 15 to about 50% w/w, still more preferably from about 20 to about 48% w/w and most particularly preferably from about 30 to about 45% w/w relative to the total weight of the bleaching composition, as the persulfate, and further contains an alkalising agent which is preferably and alkali or alkaline earth silicate, more preferably sodium silicate, potassium silicate or a mixture of sodium silicate and potassium silicate.

### F)

Cosmetic agent for blonding human hair comprising (i) a sachet which forms a closed chamber containing a water-soluble polymer comprising one or more PVOH polymers, wherein the first PVOH has a lower average molecular weight M_{w} than the second PVOH polymer and/or wherein the first PVOH polymer has a lower degree of hydrolysis than the second PVOH polymer, and (ii) a bleaching composition which is contained inside the closed chamber of the sachet, wherein the bleaching composition contains sodium percarbonate or potassium percarbonate in a quantity from about 0.5 to about 14% w/w, preferably from about 1.5 to about 13% w/w, still more preferably from about 3 to about 12% w/w, particularly preferably from about 4.5 to about 11% w/w and most particularly preferably from about 6 to about 10% w/w relative to the total weight of the bleaching composition as the percarbonate, and one or more of the group of ammonium persulfate, potassium persulfate and sodium persulfate in a quantity from about 5 to about 60% w/w, preferably from about 10 to about 55% w/w, more preferably from about 15 to about 50% w/w, still more preferably from about 20 to about 48% w/w and most particularly preferably from about 30 to about 45% w/w relative to the total weight of the bleaching composition, as the persulfate, and further contains an alkalising agent which is preferably and alkali or alkaline earth silicate, more preferably sodium silicate, potassium silicate or a mixture of sodium silicate and potassium silicate, and wherein the alkalising agent is contained in the bleaching composition in a quantity from about 20 to about 50% w/w, most preferably in a quantity from about 35 to about 45% w/w, relative in each case to the total weight of the bleaching composition.

### G)

Cosmetic agent for blonding human hair comprising (i) a sachet which forms a closed chamber containing a water-soluble polymer comprising one or more PVOH polymers, wherein the first PVOH has a lower average molecular weight M_{w} than the second PVOH polymer and/or wherein the first PVOH polymer has a lower degree of hydrolysis than the second PVOH polymer, and (ii) a bleaching composition which is contained inside the closed chamber of the sachet, wherein the bleaching composition contains sodium percarbonate or potassium percarbonate in a quantity from about 0.5 to about 14% w/w, preferably from about 1.5 to about 13% w/w, still more preferably from about 3 to about 12% w/w, particularly preferably from about 4.5 to about 11% w/w and most particularly preferably from about 6 to about 10% w/w relative to the total weight of the bleaching composition as the percarbonate, and one or more of the group of ammonium persulfate, potassium persulfate and sodium persulfate in a quantity from about 5 to about 60% w/w, preferably from about 10 to about 55% w/w, more preferably from about 15 to about 50% w/w, still more preferably from about 20 to about 48% w/w and most particularly preferably from about 30 to about 45% w/w relative to the total weight of the bleaching composition, as the persulfate, and further contains an alkalising agent which is preferably and alkali or alkaline earth silicate, more preferably sodium silicate, potassium silicate or a mixture of sodium silicate and potassium silicate, wherein the bleaching composition further contains a dispersing copolymer based on acrylates, wherein the dispersing copolymer preferably comprises a copolymer containing one or more acrylic acid or methacrylic acid units, wherein the dispersing copolymer is more preferably a methacrylic acid/methyl(meth)acrylate copolymer or an acrylic acid/methyl(meth)acrylate copolymer.

Features relating to preferred embodiments of the first object of the present disclosure which are only described with reference thereto in the preceding text are naturally also applicable correspondingly as features of preferred embodiments for the second and third objects.

The following examples are intended to explain the object of the present disclosure without in any way being limiting thereof.

### EXAMPLES

1. Formulations

The following formulations were prepared (unless otherwise indicated the quantities indicated correspond to percentage by weight

### Blonding Powder

| | Ex. 1 | Ex. 2 | Ex. 3 |
|---|---|---|---|
| Ammonium persulfate | 10.0 | 10.0 | 10.0 |
| Potassium persulfate | 28.0 | 28.0 | 28.0 |
| Sodium persulfate | 2.0 | 2.0 | 2.0 |
| Sodium silicate | 39.0 | 39.0 | 39.0 |
| (molar ratio SiO₂/Na₂O = 2.65) | | | |
| Methyl methacrylate, methacrylic acid | 1.0 | 1.0 | 1.0 |
| Copolymer (Degalan RG S hv, | | | |
| Evonic) | | | |
| Carboxymethyl cellulose (Na salt) | 1.5 | 1.5 | 1.5 |
| (Cekol 50000, CP Kelco) | | | |
| Hydroxyethyl cellulose | 2.5 | 2.5 | 2.5 |
| (Natrosol 250 HR, Ashland) | | | |
| Silica gel, hydrophilic | 0.2 | 0.2 | 0.2 |
| Polyquaternium-4 | 0.3 | 0.3 | 0.3 |
| EDTA, tetrasodium salt | 1.6 | 1.6 | 1.6 |
| Sodium percarbonate | 2.0 | 4.0 | 8.0 |
| Magnesium carbonate | 11.9 | 9.9 | 5.9 |

The preparations for examples 1 to 3 were packaged in a sachet (single-chamber container, pouch) made from a water-soluble film supplied by company Monosol. The water-soluble film used was the film SCP 20633 (Monosol, Film SCP 20633: water-soluble film, containing a PVOH polymer, degree of hydrolysis approx. 89%, average molecular weight=95,000 g/mol).

| | Ex. 4 | Ex. 5 | Ex. 6 |
|---|---|---|---|
| Ammonium persulfate | 10.0 | 10.0 | 10.0 |
| Potassium persulfate | 28.0 | 28.0 | 28.0 |
| Sodium persulfate | 2.0 | 2.0 | 2.0 |
| Sodium silicate | 39.0 | 39.0 | 39.0 |
| (molar ratio SiO₂/Na₂O = 2.65) | | | |
| Methyl methacrylate, methacrylic acid | 1.0 | 1.0 | 1.0 |
| Copolymer (Degalan RG S hv, | | | |
| Evonic) | | | |
| Carboxymethyl cellulose (Na salt) | 1.5 | 1.5 | 1.5 |
| (Cekol 50000, CP Kelco) | | | |
| Hydroxyethyl cellulose | 2.5 | 2.5 | 2.5 |
| (Natrosol 250 HR, Ashland) | | | |
| Silica gel, hydrophilic | 0.2 | 0.2 | 0.2 |
| Polyquaternium-4 | 0.3 | 0.3 | 0.3 |
| EDTA, tetrasodium salt | 1.6 | 1.6 | 1.6 |
| Sodium percarbonate | 2.0 | 4.0 | 8.0 |
| Magnesium carbonate | 11.9 | 9.9 | 5.9 |

The preparations for examples 4 to 6 were packaged in a sachet (single-chamber container, pouch) made from a water-soluble film supplied by company Monosol. The water-soluble film used was the film LX 9643 (Monosol, Film LX 9643: water-soluble film, containing polyvinyl alcohol/polyvinyl acetate copolymer, degree of hydrolysis approx. 84%, average molecular weight=39,000 g/mol).

2. Application

The sachets (single-chamber container, pouch) were each mixed with lukewarm water in a ratio of 1:2 and homogenised. The film was dissolved and the formulation was released. The homogenised mixtures were applied to light brown Fischbach & Miller Hair and left to take effect for 45 minutes, after which they were rinsed out with tap water. Then the hair was dried. The hair could be combed easily and had a soft texture.

3. Blonding Effect

Examples 1 to 3 were evaluated in terms of their blonding effect:

The hair was measured colorimetrically before and after the application with a spectral photometer produced by Datacolor (SF450), and the colour distance (ΔE) was determined.

| | L | a | b | ΔE value |
|---|---|---|---|---|
| Before blonding | 23.75 | 6.64 | 10.78 | — |
| Ex. 1 | 39.86 | 11.42 | 25.25 | 22.17 |
| Ex. 2 | 43.33 | 10.72 | 25.50 | 24.84 |
| Ex. 3 | 43.91 | 11.38 | 26.93 | 26.26 |

The L value indicates the lightness of the colour (L=0, black; L=100, white). The higher the L value of the treated hair, the greater the lightening effect was on the hair.

The ΔE value used to evaluate the colour intensity is derived from the L*a*b* colorimetric values as follows:

ΔE=[(Lᵢ−L₀)²+(aᵢ−a₀)²+(bᵢ−b₀)]^{1/2}

L₀, a₀ and b₀: colorimetric values before blonding
Lᵢ, aᵢ and bᵢ: colorimetric values after blonding

The ΔE value indicates the colour difference between the untreated hair sample and the same hair sample after treatment. The larger the ΔE value is, the greater the colour difference is (i.e., the colour distance) between the undyed and the dyed skin, and accordingly the stronger the blonding effect is. In the experiments, it was found that increasing the quantity of peroxide does not strengthen the blonding effect proportionally.

4. Storage Tests

In order to determine storage stability, the pouches (sachets) of example 1 to 6 were stored at room temperature for 4 weeks. Then, the pouches were inspected and evaluated in terms of their impermeability, brittleness and chemical resistance.

| | | | Chemical |
|---|---|---|---|
| | Impermeability | Brittleness | resistance |
| Ex. 1, Film Monosol SCP 20633 | 1 | 1 | 1 |
| Ex. 2, Film Monosol SCP 20633 | 1 | 1 | 1 |
| Ex. 3, Film Monosol SCP 20633 | 1 | 1 | 1 |
| Ex. 4, Film Monosol LX 9643 | 1 | 2 | 3 |
| Ex. 5, Film Monosol LX 9643 | 1 | 2 | 3 |
| Ex. 6, Film Monosol LX 9643 | 1 | 2 | 3 |

1 = Very good
3 = Acceptable
6 = Very poor

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for blonding human hair, comprising
(i) a sachet forming a closed chamber, wherein the sachet comprises a water-soluble polymer, and
(ii) a bleaching composition which is included inside the closed chamber of the sachet, wherein the bleaching composition comprises:
at least one percarbonate, and
at least on persulfate.

2. The cosmetic agent according to claim 1, wherein the water-soluble polymer comprises one or more polyvinyl alcohol polymers.

3. The cosmetic agent according to claim 2, wherein the water-soluble polyvinyl alcohol polymer has an average molecular weight M_{w} from about 30,000 to about 170,000 g/mol.

4. The cosmetic agent according to claim 1 wherein the water-soluble polymer comprises one or more polyvinyl alcohol polymers constituting at least 70% w/w relative to the total weight of the sachet.

5. The cosmetic agent according to claim 1 wherein the water-soluble polymer further comprises a polyvinyl alcohol copolymer (PVOH copolymer) and/or a polysaccharide selected from the group of methyl cellulose, carboxymethyl cellulose, ethyl cellulose, hydroxyethyl cellulose, hydroxypropyl cellulose, hydroxypropylmethyl cellulose, dextrin and hydroxypropyl starch.

6. The cosmetic agent according to claim 1 wherein the bleaching composition comprises sodium percarbonate or potassium percarbonate as the percarbonate in a quantity from about 0.5 to about 14% w/w relative to the total weight of the bleaching composition as the percarbonate, and one or more of the group of ammonium persulfate, potassium persulfate and sodium persulfate as the persulfate in a quantity from about 5 to about 60% w/w relative to the total weight of the bleaching composition.

7. The cosmetic agent according to claim 1 wherein the bleaching composition further comprises an alkalising agent chosen from an alkali or alkaline earth silicate.

8. The cosmetic agent according to claim 7, wherein the alkalising agent is included in the bleaching composition in a quantity from about 20 to about 50% w/w relative to the total weight of the bleaching composition.

9. The cosmetic agent according to claim 1 wherein the bleaching composition comprises as the persulfate a mixture of ammonium persulfate, potassium persulfate and sodium persulfate, in which ammonium persulfate is included in a quantity from about 1 to about 15% w/w, potassium persulfate in a quantity from greater than 0 to about 45% w/w and sodium persulfate in a quantity from greater than 0 to about 5% w/w, subject to the condition that the sum of the quantities of ammonium persulfate, potassium persulfate and sodium persulfate is between about 15 to about 65% w/w, wherein the bleaching composition comprises sodium percarbonate as the percarbonate in a quantity from about 1 to about 10% w/w, and wherein the bleaching composition comprises sodium silicate as an alkalising agent in a quantity from about 35 to about 45% w/w, wherein the quantities are each relative to the total weight of the bleaching composition.

10. The cosmetic agent according to claim 1 wherein the bleaching composition further comprises a dispersing copolymer having one or more acrylic acid or methacrylic acid units.

11. The cosmetic agent according to claim 1 wherein the bleaching composition further comprises a thickener, which is selected from the group of a carboxymethyl cellulose, the Na salt thereof and hydroxymethyl cellulose.

12. The cosmetic agent according to claim 1 wherein when dissolved or suspended in a double quantity of water—relative to the weight of the bleaching composition—the bleaching composition has a pH value from about 8 to about 12.

13. A method for blonding human hair, in which
(a) the cosmetic agent according to claim 1 is introduced into a quantity of water,
(b) the mixture obtained from (a) is homogenised, and
(c) the homogenised mixture is applied to the human hair.

14. (canceled)

15. The cosmetic agent according to claim 2, wherein the water-soluble polymer has an average molecular weight M_{w} from about 60,000 to about 110,000 g/mol.

16. The cosmetic agent according to claim 1 wherein the water-soluble polymer further comprises a polysaccharide comprising a hydroxypropyl starch.

17. The cosmetic agent according to claim 1 wherein the bleaching composition comprises sodium percarbonate or potassium percarbonate as the percarbonate in a quantity from about 6 to 10% w/w relative to the total weight of the bleaching composition.

18. The cosmetic agent according to claim 1 wherein the bleaching composition comprises one or more of the group of ammonium persulfate, potassium persulfate and sodium persulfate as the persulfate in a quantity from about 30 to about 45% w/w relative to the total weight of the bleaching composition.

19. The cosmetic agent according to claim 1 wherein the bleaching composition further comprises an alkalising agent chosen from sodium silicate, potassium silicate or a mixture of sodium silicate and potassium silicate.

20. The cosmetic agent according to claim 1 wherein the bleaching composition further comprises a dispersing copolymer chosen from a methacrylic acid/methyl(meth)acrylate copolymer or an acrylic acid/methyl(meth)acrylate copolymer.

21. A cosmetic agent for blonding human hair, comprising
(i) a sachet forming a closed chamber, wherein the sachet comprises a water-soluble polymer wherein the water-soluble polymer comprises one or more polyvinyl alcohol polymers having an average molecular weight M_{w} from about 30,000 to about 170,000 g/mol, and
(ii) a bleaching composition which is included inside the closed chamber of the sachet, wherein the bleaching composition comprises:
sodium percarbonate or potassium percarbonate in a quantity from about 6 to 10% w/w relative to the total weight of the bleaching composition,
sodium persulfate in a quantity from about 30 to about 45% w/w relative to the total weight of the bleaching composition,
an alkalising agent chosen from sodium silicate, potassium silicate or a mixture of sodium silicate and potassium silicate, wherein the alkalising agent is present in a quantity from about 35 to about 45% w/w relative to the total weight of the bleaching composition.
